(19) United States
(12) Patent Application Publication
(10) Pub. No.: US 2018/0184892 A1
(43) Pub. Date: Jul. 5, 2018

(54) ENDOSCOPE AND METHOD OF USE

(71) Applicant: Meditrina, Inc., Cupertino, CA (US)

(72) Inventors: Csaba Truckai, Saratoga, CA (US); Daniel Truckai, Saratoga, CA (US); Britta Nelson, Loomis, CA (US); Nicholas Landgraf, Huntington Woods, MI (US)

(73) Assignee: Meditrina, Inc., Cupertino, CA (US)

(21) Appl. No.: 15/861,474

(22) Filed: Jan. 3, 2018

Related U.S. Application Data
(60) Provisional application No. 62/442,120, filed on Jan. 4, 2017.
(60) Provisional application No. 62/442,805, filed on Jan. 5, 2017.
(60) Provisional application No. 62/443,377, filed on Jan. 6, 2017.

(51) Int. Cl. A61B 1/303; A61B 1/00; A61B 1/015

(52) U.S. Cl. CPC A61B 1/303; A61B 1/00119; A61B 1/307; A61B 1/015; A61B 1/0014; A61B 1/00009

(57) ABSTRACT

Endoscope systems include an endoscope shaft assembly which comprises a shaft having a working channel, an inflow channel configured to be coupled to a fluid source, and an outflow channel configured to be coupled to a negative pressure source. A hub is coupled to a proximal portion of the shaft, and an image sensor is disposed on a distal portion of the shaft. A control unit is configured to adjust both a fluid inflow from the fluid source through the inflow channel to the working space and a fluid outflow to the negative pressure source through the outflow channel from the working space. A handle assembly is detachably connected to the hub of the endoscope shaft assembly, and the handle assembly comprises a control pad having at least one actuator which may be wired or wirelessly linked to the controller for adjusting fluid inflows and outflows through the inflow channel and outflow channel in the shaft.

### CROSS-REFERENCE TO RELATED APPLICATIONS

This application claims the benefit of Provisional No. 62/442,120 (Attorney Docket No. 50553-706.101), filed Jan. 4, 2017, Provisional No. 62/442,805 (Attorney Docket No. 50553-707.101), filed Jan. 5, 2017, and Provisional No. 62/443,377 (Attorney Docket No. 50553-708.101), filed Jan. 6, 2017, the entire contents of which are incorporated herein by reference.

### BACKGROUND OF THE INVENTION

### 1. Field of the Invention

The present invention relates to an endoscope assembly, and more particularly in endoscope with a working channel for use in hysteroscopy, and a method of use.

Endoscopes are used in a wide variety of minimally invasive surgical procedures, including laparoscopy, arthroscopy, and the like. Of particular interest to the present application, hysteroscopy is a minimally invasive procedure for resecting fibroids and performing similar interventions in a patient's uterus. Hysteroscopy utilizes a hysteroscope which is a type of endoscope that carries optics for viewing, a light source for illumination, and a working channel. Interventional tools, such as an electrosurgical loop or other cutter, forceps, and the like, are introduced though the working channel of the hysteroscope to perform the hysteroscopy while the patient's uterus is insufflated. The hysteroscope is typically introduced through a passage in a transcervical sheath which also allows insufflation of the uterine cavity.

In performing hysteroscopy and other endoscopic procedures, the physician is often challenged with controlling many variables, including inflation pressure in the uterus or other work space, the inflow and outflow of fluids to and from the workspace, illumination of the workspace, all while simultaneously manipulating the interventional tools and viewing the procedure on a remote image display. Present endoscopic systems often have control functions located on controllers and the video display units may also be located at positions which require the physician to look away from the patient and endoscope during a procedure.

For these reasons, it would be desire able to provide endoscopic systems which are convenient and simple to use during hysteroscopic and other endoscopic procedures. In particular, such endoscopic systems should allow a physician to perform procedures with minimal distractions caused by the need to make system adjustments during the procedure. The endoscopic systems will preferably provide user interface components on reusable system assemblies while routing fluid flows through disposable system assemblies. At least some of these objectives will be met by the inventions described and claimed hereinafter.

### 2. Background of the Related Art

Related commonly owned US patent applications include Ser. Nos. 15/712,603 and 15/836,460, the full disclosures of which are incorporated herein by reference.

### BRIEF SUMMARY OF THE INVENTION

The present invention provides endoscope systems which include separate endoscope, handle, and control assemblies which may be interconnected in ways that afford convenient and economic patient treatment. In particular, the endoscope assembly can be fabricated at a low cost and may be disposable. The handle assembly and control unit will usually include the higher cost components and may be reusable. Most system functions may be controlled by the user from a control pad on the handle which is connected to the control unit by a wireless or wired link. The control unit provides fluid and pressure control, and all fluid lines may be directly connected to the endoscope, bypassing the handle assembly.

Endoscope systems of the present invention may comprise an endoscope shaft assembly which comprises a shaft having a working channel, an inflow channel configured to be coupled to a fluid source, and an outflow channel configured to be coupled to a negative pressure source. A hub may be coupled to a proximal portion of the shaft, and an image sensor may be disposed on a distal portion of the shaft. A control unit may be configured to adjust a fluid inflow from the fluid source through the inflow channel to the working space and a fluid outflow to the negative pressure source through the outflow channel from the working space. A handle assembly may be detachably connected to the hub of the endoscope shaft assembly, and the handle assembly may comprise a control pad having at least one actuator which may be wired or wirelessly linked to the controller for adjusting fluid inflows and outflows through the inflow channel and outflow channel in the shaft.

The endoscope systems of the present invention may further comprise an image display coupled to the handle assembly or remote from the handle, an in some casesbeing detachably coupled to the handle assembly. The endoscope systems may still further comprise an image processor, where the image processor may disposed in the control unit and may be electronically coupled to both the image sensor in the shaft assembly and the image display in the handle assembly. The image processor may alternatively be disposed in the handle assembly and be electronically coupled to both the image sensor in the shaft assembly and the image display in the handle assembly. Additionally or alternatively, an image display may be disposed on the control unit and be interconnected with the image sensor and image processor as noted above.

In other aspects of the present invention, the hub may have a first port for detachable connection to the fluid source and a second port for detachable connection to the negative pressure source so that no fluids flow through the handle, thus facilitating cleaning and reuse of the handle. The endoscope system may still further comprise one or more tubular connectors (typically disposable) for detachably connecting the first and second ports on the hub to the control unit, where the control unit may comprise a first peristaltic pump operatively connected to the fluid source for delivering fluid inflows to the to the inflow channel and a second peristaltic pump for aspirating fluid outflows from the outflow channel.

In still other aspects of the present invention, the endoscope systems may further comprise a pressure sensor. The pressure sensor may be disposed within the endoscope component assembly. Alternatively or additionally, the pressure sensor may be operatively connected to a flow path between the fluid source and the inflow channel in the shaft. Alternatively or additionally, the pressure sensor may be operatively connected to a flow path between the outflow channel in the shaft and the negative pressure source.

In further aspects of the present invention, the distal portion of the endoscope shaft may carry at least one illumination element. The at least one illumination element may comprise at least one light emitting diode (LED), and at least one actuator in the handle assembly may be configured to adjust light intensity of the at least one LED.

In yet other aspects of the present invention, the controller may be configured to maintain fluid pressure at a set pressure in the working space, and at least one actuator in the handle assembly may be configured to adjust the set pressure.

In additional aspects of the present invention, at least one actuator in the handle assembly may be configured to capture still video images from the image sensor. Alternatively or additionally, at least one actuator in the handle assembly may be configured to capture video clips from the image sensor, where optionally a video processor may be located in the handle component for processing video signals from the image sensor.

In some particular embodiments, a first electrical connector in the hub may be adapted for detachable coupling to a second connector in the handle component for carrying video and control signals. Often, a third electrical connector in the hub that is adapted for detachable coupling to a fourth connector in the handle component for connecting an electrical source to the at least one LED.

### BRIEF DESCRIPTION OF THE DRAWINGS

Additional aspects of the invention will become clear from the following description of an illustrative embodiment and from the attached drawings, in which:

FIG. 1 is a perspective view of an embodiment of an endoscope corresponding to the invention.

FIG. 2 is a perspective view of a distal portion of the endoscope shaft including a resilient, elastomeric distal portion that carries an image sensor and LEDs, showing the distal portion in a straight insertion configuration.

FIG. 3 is a perspective view sectional view of a portion of the shaft in phantom view, the elastomeric distal portion and the image sensor of FIG. 2 taken along line 3-3 of FIG. 2 in the straight insertion configuration of FIG. 2.

FIG. 4A is a longitudinal sectional view through a portion of the shaft and the distal elastomeric portion of the endoscope in an insertion configuration.

FIG. 4B is another longitudinal sectional view similar to that of FIG. 4A with the distal elastomeric portion in a deformed or displaced configuration after being deflected by a rigid tool shaft inserted through a working channel in the endoscope.

FIG. 5A illustrates a method of use of the endoscope of FIGS. 1-4B in a hysteroscopy wherein a cervical sealing assembly is provided and thereafter the endoscope shaft is introduced through the seal assembly into a patient's uterine cavity in an insertion configuration as shown in FIGS. 1, 2, 3 and 4A further showing the field of view of the image sensor.

FIG. 5B illustrates a subsequent step of the hysteroscopy method of FIG. 5A wherein a treatment tool is introduced through the endoscope shaft which deflects the distal elastomeric portion to provide the deployed or displaced configuration.

FIG. 6 is a perspective view of an alternative embodiment of an endoscope corresponding to the invention with the endoscope shaft again including a resilient, elastomeric distal portion that carries an image sensor and LEDs.

FIG. 7 is an exploded view of the components of the endoscope of FIG. 6.

FIG. 8 is a side view of the endoscope of FIGS. 6-7 with the distal elastomeric portion in a deformed configuration after being deflected by a biopsy tool shaft inserted through a working channel.

FIG. 9 is a perspective view of the handle and control panel of the endoscope of FIGS. 6-7.

FIG. 10 is a perspective view of a fluid management system of the invention that is functionally coupled to the endoscope of FIGS. 6-7.

FIG. 11 is a schematic view of components of the fluid management system of FIG. 10.

FIG. 12 is an enlarged schematic view of a flow sensor fitting in an outflow line of the fluid management system of FIG. 10.

### DETAILED DESCRIPTION OF THE INVENTION

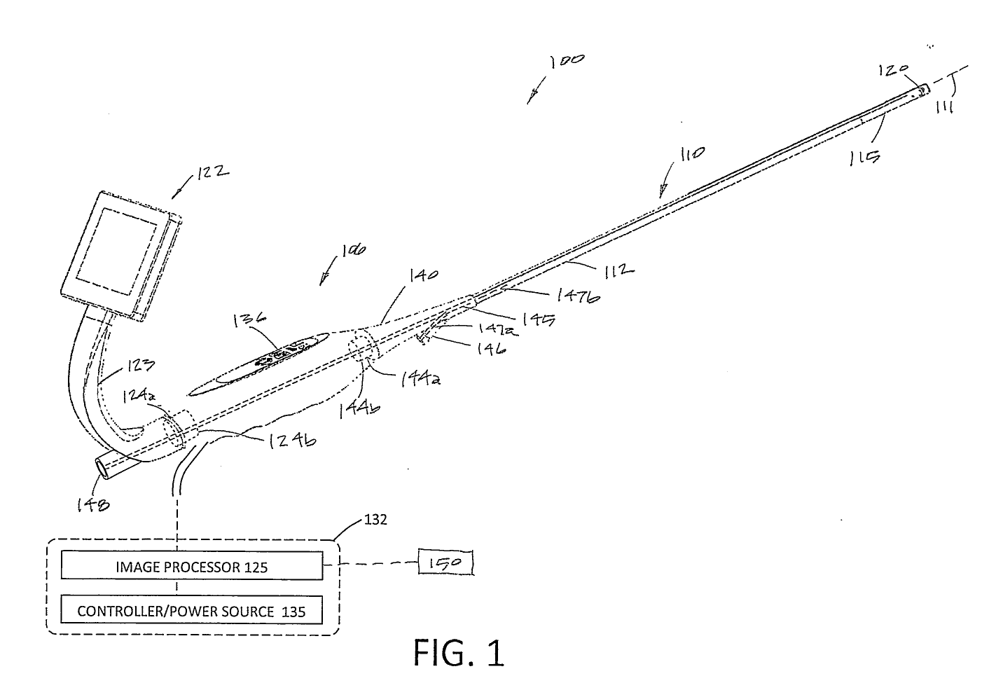

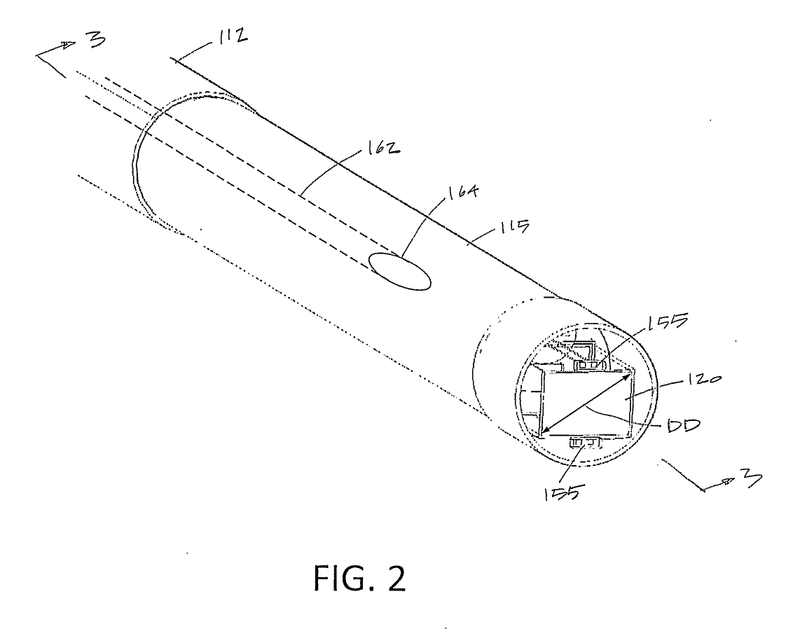

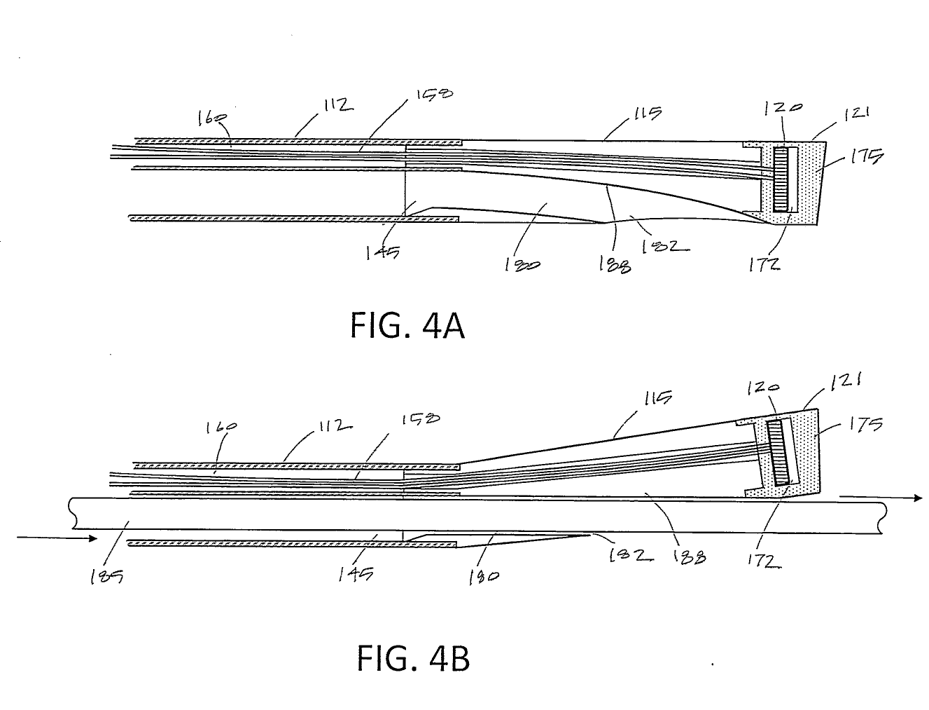

FIG. 1 illustrates an endoscope endoscope 100 corresponding to the invention which comprises a proximal handle portion 106 is coupled to a shaft portion 110 extending along longitudinal axis 111. The shaft includes a rigid proximal portion 112 that extends to a flexible, resilient housing or elastomeric distal portion 115. An electronic image sensor 120 is carried in the elastomeric distal portion 115 of the shaft as shown in FIG. 2. The image sensor 120 is covered by a transparent tip member 121 (not shown in FIG. 2) that can be seen in FIG. 4A. The transparent tip 121 can further comprise a focusing lens and/or a prism for modifying the sensor's field of view. In one variation, the handle 106 carries a detachable image display 122 that has coupling member 123 configured with a display connector 124a that mates with handle connector 124b. The image sensor 120 is further operatively connected to an image processor 125 carried in a remote base unit 132 together with a controller/power source 135 for the sensor 120 and LEDs described below. Alternatively, the image processor 125 or components thereof can be carried in the handle 106. A control pad 136 is provided in the handle with actuator buttons for operating the system and image sensor, for example to turn on/off the image sensor 120, capture still images, adjust light from LEDs, etc.

In one variation, the shaft 110 extends distally from a hub 140 that is detachably coupled to handle 106 wherein hub connecter 144a mates with handle connector 144b. In some variations, the shaft 110 may be rotated while the handle 106 is adapted for being held in a stable position. Thus, the handle 106 and display 122 can positioned at a selected angle by the physician, and the shaft 110 can be rotated to orient the image sensor 120 in a selected rotational direction when in use. Such rotation can be accomplished by a rotating grip (not shown) in the hub 140 or in the shaft adjacent the hub 140.

In one variation, shaft 110 has a diameter ranging between 2.5 mm and 10 mm with a length configured for use in hysteroscopy. More commonly, the shaft diameter is from 4 mm to 6 mm in diameter.

As will be described below, the handle 106 and shaft 110 are configured with a working channel 145 that may have a diameter ranging between 1 mm and 6 mm. The working channel or tool-receiving channel 145 is adapted for receiving various types of tools. For example, a biopsy device may have a flexible shaft (not shown) with a diameter ranging from 1 mm to 3 mm and can be introduced through port 146 on the hub 140 which extends through a curved path 147a to a straight channel 147b in the shaft 110. Alternatively, a tissue resecting device (not shown) can be used which may have a larger rigid shaft with a diameter, for example, from 2.5 mm to 5 mm. Such a rigid shaft tool may be introduced through port 148 in display coupling member 123 and handle as shown in FIG. 1. The endoscope or the endoscope shaft 110 may be disposable or re-usable. In one variation, the shaft portion 110 is disposable as described above and is detachable from the handle 106 which is reusable.

As can be seen in one variation in FIG. 1, the display 122 is adapted for detachable coupling to the handle 106. In another system variation, the display 122 can be remote and does not have to be attached to handle 106 and the image processor 125 controller 135 can send images signals to a remote display 150 (see FIG. 1) such as a monitor in an endoscopic viewing and recording system as is known in the art.

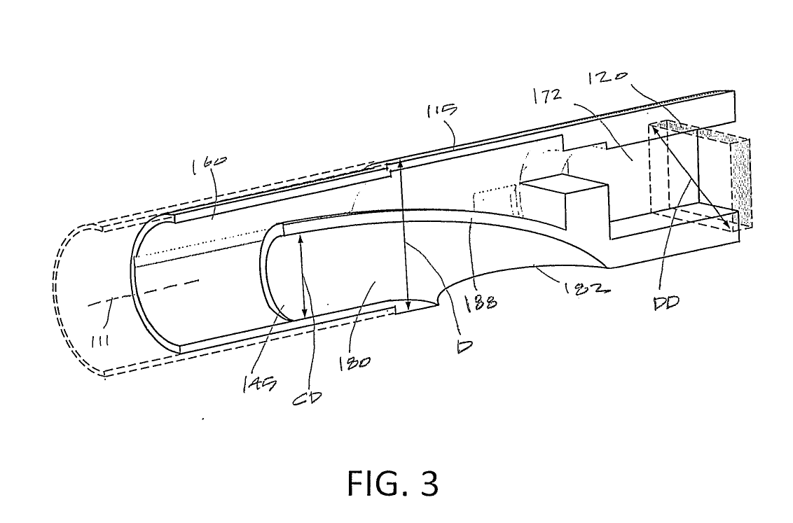

Now turning to FIGS. 2 and 3, the distal elastomeric portion 115 also carries one or more light emitters, for example, LEDs indicated at 155. The image sensor 120 can be coupled to the image processor 125 by wire leads 158 (FIGS. 4A and 4B) which can be independent wires or an elongated flex circuit extending through passageway 160 in the shaft 110 and elastomeric portion 115. Similarly, wire leads (not shown) connect the LEDs to the remote electrical source and controller 140.

Still referring to FIG. 2, it can be seen that a flow channel 162 extends through the shaft 110 and has an open termination 164 in the distal elastomeric portion 115. Such a flow channel 162 can be used for either fluid inflows or fluid outflows from a working space or for measuring pressure in the working space with a static fluid channel. The proximal end of the flow channel 162 can communicate with a Luer fitting in the housing 140 (not shown). It should be appreciated that first and second flow channels with open distal terminations can be provided, with Luer connections in the hub 140 as just described with such channels being used for more the one of the purposes described above. In one variation, a fluid management system can be coupled to inflow and outflow channels in the endoscope to provide a circulating flow through a patient's uterine cavity and can maintain a set intra-cavity pressure as is known in the art.

Now referring to FIG. 3, a longitudinal sectional view of the elastomeric distal portion 115 or housing is shown. The image sensor 120 is carried in a distal region of the housing 115. A transparent distal tip 121 is shown in FIG. 4A that can comprise a clear material such as a plastic lens material which is sealed and coupled to the distal end of the housing 115. The elastomeric portion 115 and transparent tip 121 coupled together to provide a space 172 therein that carries the image sensor 120. In a variation shown in FIGS. 4A-4B, it can be seen that the transparent distal tip 121 further comprises a prism 175 for altering the direction of the field of view of the image sensor 120 as will be described below.

Referring again to FIG. 3, the endoscope shaft 110 and elastomeric portion 115 is shown in an insertion profile or configuration wherein the elastomeric distal portion 115 is in a repose, non-tensioned position and the working channel 145 has a distal portion 180 that is curved with an open termination 182 in the side or bottom of the elastomeric portion 115. FIG. 4A shows the elastomeric portion 115 in another schematic view again in the straight insertion configuration. In FIG. 4B, it can be seen that when the physician inserts a rigid tool shaft 185 through the working channel 145 it will interface with the wall 188 of the repose, curved working channel portion 180 in the elastomeric portion 115. Continued advancement of the tool shaft 185 through the working channel 145 and curved repose channel portion 180 will cause the curved channel portion 180 to straighten until the working end of the tool exits the open termination 182 of the working channel 145. In other words, elastomeric portion 115 is deformed or displaced to a tensioned position wherein the image sensor 120 is moved away from the longitudinal axis of the shaft 110. When the shaft 185 of the tool is withdrawn from the working channel, the elastomeric portion 115 will return from the tensioned position of FIG. 4B to the repose or non-tensioned position of FIG. 4A.

In general, the endoscope corresponding to the invention allows for the use of an image sensor 120 having a large diagonal dimension relative to the insertion profile of the endoscope shaft 110 while at the same time providing a working channel 145 that has a large channel diameter CD relative to the insertion profile of the endoscope shaft 110. More in particular, the endoscope comprises a shaft having a shaft diameter SD extending about a longitudinal axis 111 to a distal housing 115, an image sensor with a diagonal dimension DD carried by the distal housing 115, and a working channel having a diameter CD extending through the shaft and distal housing, wherein the channel portion in the distal housing is adjustable in shape to accommodate a tool introduced therethrough and wherein the combined sensor's diagonal dimension DD and the channel diameter CD is greater than the shaft diameter SD (see FIG. 3). In a variation, the sensor diagonal dimension DD is greater than 50% of the shaft diameter SD, greater than 60% of the shaft diameter or greater than 70% of the shaft diameter. In a variation, the working channel diameter CD is greater than 30% of the shaft diameter, greater than 40% of the shaft diameter or greater than 50% of the shaft diameter. In other words, the working channel portion in the distal housing is adjustable between a curved shape and a straight shape. In another variation described below, the channel portion in the distal housing is adjustable between an at least partially collapsed shape and a non-collapsed shape.

In another aspect of the invention, the image sensor 120 can be carried in a non-orthogonal position relative to the longitudinal axis of the shaft 110 to orient the sensor's field of view to be aligned with a working space distal from the end of the endoscope after a tool is inserted through the working channel 145. In a variation, the image sensor 120 can be carried by the elastomeric portion 115 at an angle ranging between 45° to 90° relative to the longitudinal axis 111 of the proximal shaft portion 112 to provide a selected field of view.

In another aspect of the invention, the endoscope comprises a shaft extending about a longitudinal axis to a distal housing, an image sensor 120 carried by the distal housing 115 and a working channel 145 extending through the shaft and distal housing wherein a portion of the housing proximate the image sensor and the working channel comprises a shape-adjustable component or wall 188 as shown in FIG. 3. The shape-adjustable component 188 comprises at least one of an elastomeric material, a flexible material and a hinged component. The endoscope shaft 110 and distal housing 115 have a straight cylindrical shape for insertion into a patients' body and is capable of adjustment to a non-straight shape for accommodating a tool introduced through the working channel 145. In a variation, the portion of the working channel 180 in the distal housing 115 is adjustable between a non-straight shape and a straight shape (see FIG. 3). The elastomeric distal housing 115 has a repose position in which the working channel 145 has a non-straight shape and a tensioned position wherein the working channel has straight shape for accommodating a tool introduced therethrough. In a variation, the diagonal of the image sensor is greater than 50% of the cross-section of the shaft and the diameter of the working channel is greater than 50% of the cross-section of the shaft.

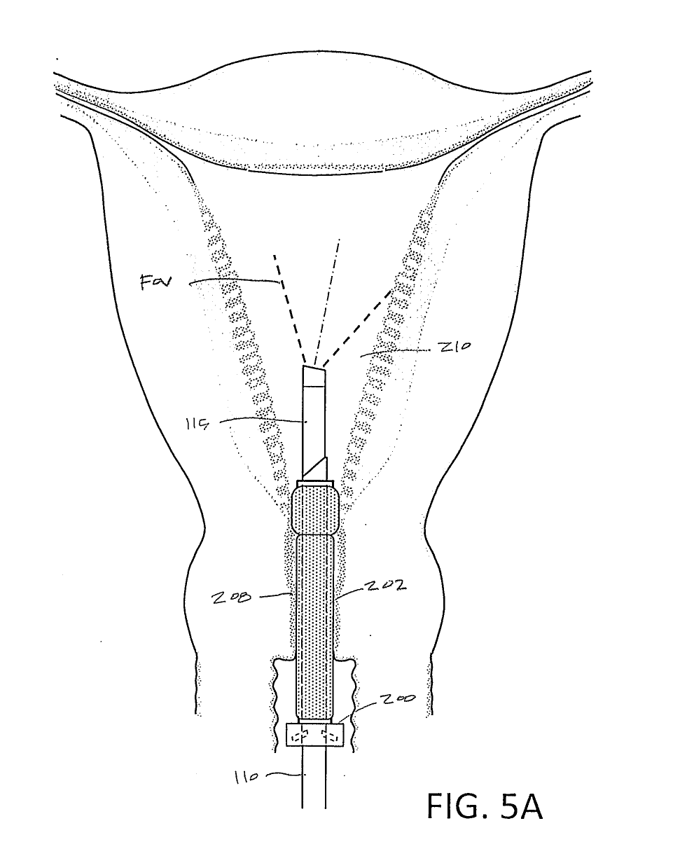

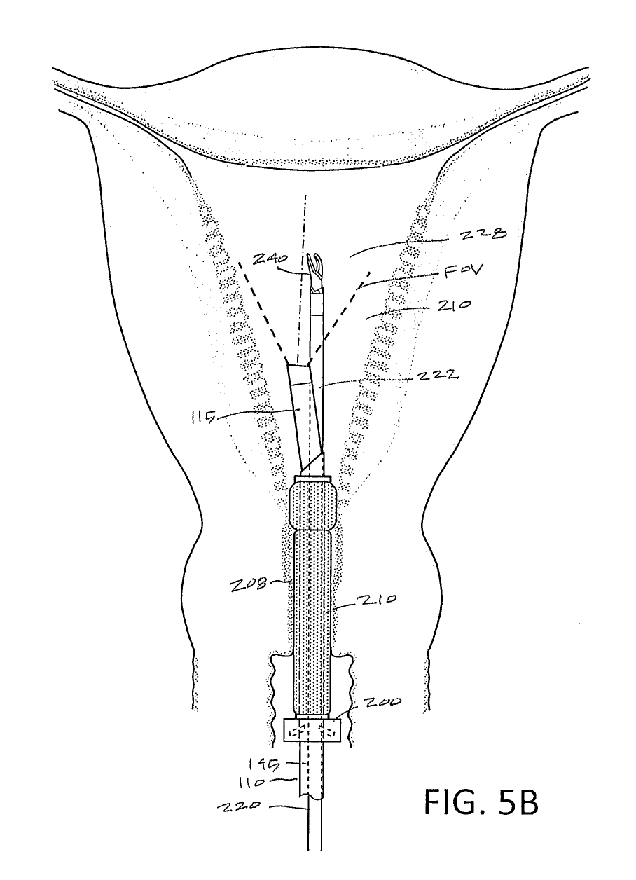

FIG. 5A illustrates a method of the invention to carry out a planned hysteroscopic procedure, wherein an introducer 200 with a cervical seal structure 202 is inserted into the patient's endocervical canal 208 to access the uterine cavity 210. The cervical seal 202, for example, can be a balloon that is expanded to provide an occlusive seal. Other types of cervical seals are known in the art and may be used such as foams, plugs, a seal member with elastomeric fins and the like. After positioning the seal 202 in the endocervical canal 208, the physician then may use a fluid management system adapted for use with inflow and outflow channels (not shown) through the introducer 200 for distending the uterine cavity. A typical fluid management system may provide a circulating flow through the patient's uterine cavity 210 and also to maintain a set fluid pressure therein.

Thereafter, the endoscope 100 and display 122 are assembled (see FIG. 1) and coupled to the controller 135. Next, still referring to FIG. 5A, the endoscope shaft 110 is introduced through the introducer 200 so that the elastomeric distal portion 115 of the endoscope is positioned in the patient's uterine cavity 210. The physician then may examine the patient's uterine cavity and diagnose any abnormalities.

In one example, the physician may identify abnormal tissue in the uterine cavity 210, such as adhesion, polyp or submucosal fibroid. The physician then may elect to treat the abnormal tissue with a suitable tool that can be introduced through the working channel 145 in the endoscope 100. In one example shown in FIG. 5B, the physician elects to use a scissor-like tool 220 for resecting an adhesion or a polyp. The tool 220 has a shaft 222 which may be rigid and has a diameter ranging from 2.5 mm to 5 mm that is configured for mechanical cutting or resection of tissue. As can be seen in FIG. 5B, the introduction of the rigid shaft 222 of the resection tool 220 through the working channel 145 causes deflection of the elastomeric distal portion 115 to thus provide a straight pathway through the endoscope shaft 110 past the deflected elastomeric portion 115 to a working space indicated at 228.

FIG. 5B also illustrates that the field of view FOV of the image sensor 120 and prism 175 is oriented so that the working end 240 of the tool 220 and the working space 228 is effectively in the center of such a field of view FOV.

In general, an endoscope of the invention comprises an elongated member extending about a longitudinal axis through a proximal portion and a distal elastomeric portion, an image sensor carried by the elastomeric portion wherein the elastomeric portion is aligned with the longitudinal axis in a repose configuration for introduction into a patient's body and wherein the elastomeric portion is adapted for deformation to a tensioned configuration by a tool introduced through a working channel therein. In this variation, the central axis of the working channel in the repose position is not aligned with the longitudinal axis 111 of the shaft 110. The central axis of the working channel in the elastomeric portion in the repose position diverges away from said longitudinal axis 111 in a curve or at an angle.

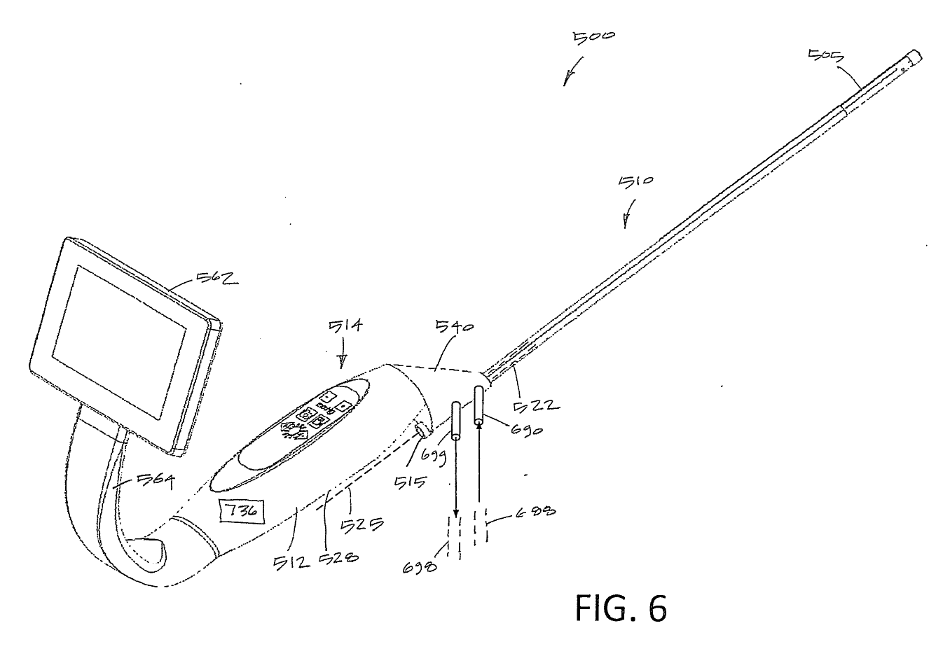

Now turning to FIG. 6, another variation of endoscope 500 is shown with a distal, deformable elastomeric portion 505. This embodiment is similar to the endoscope shown in FIG. 1 except that the introducer shaft 510 is adapted to be offset from the grip portion 512 of the handle 514. In this variation, the port 515 that is adapted to receive a tool shaft inserted into the working channel 522 is offset from the grip portion 512 of handle 514. In one variation, the central axis 525 of the working channel 522 is offset from the inferior surface 528 of the grip portion 512 of handle 514 by at least 1 cm, at least 1.5 cm, or at least 2 cm (see FIG. 8). In a typical embodiment, the central axis 525 of the working channel 522 is offset between 2 cm and 4 cm from the inferior surface 528 of handle 514.

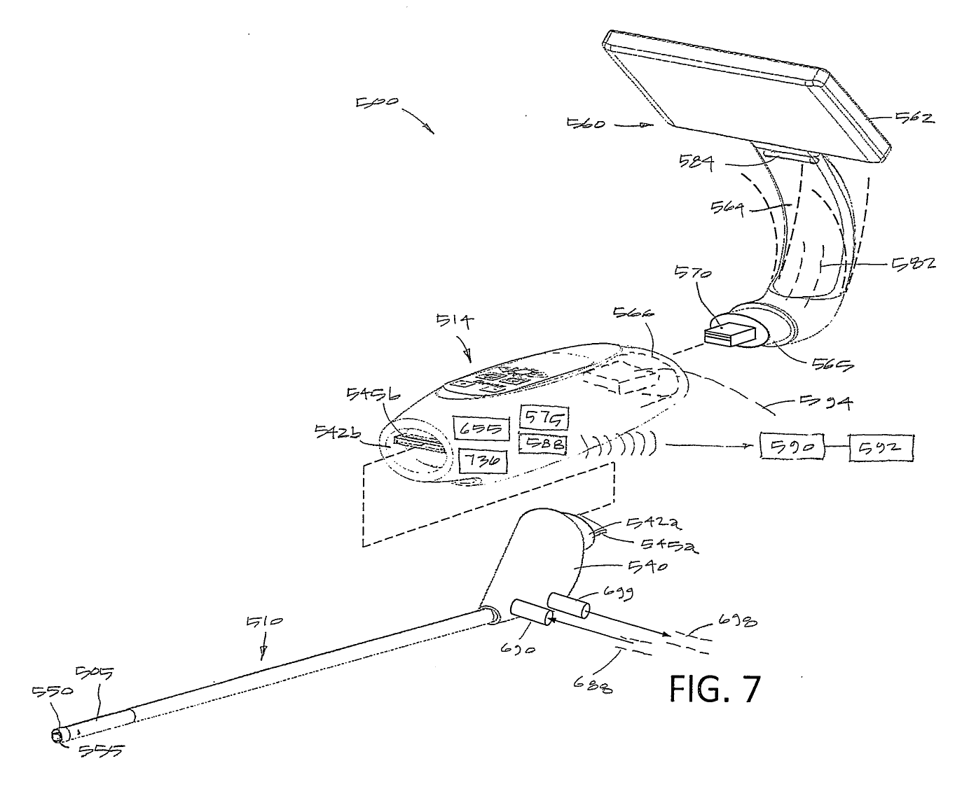

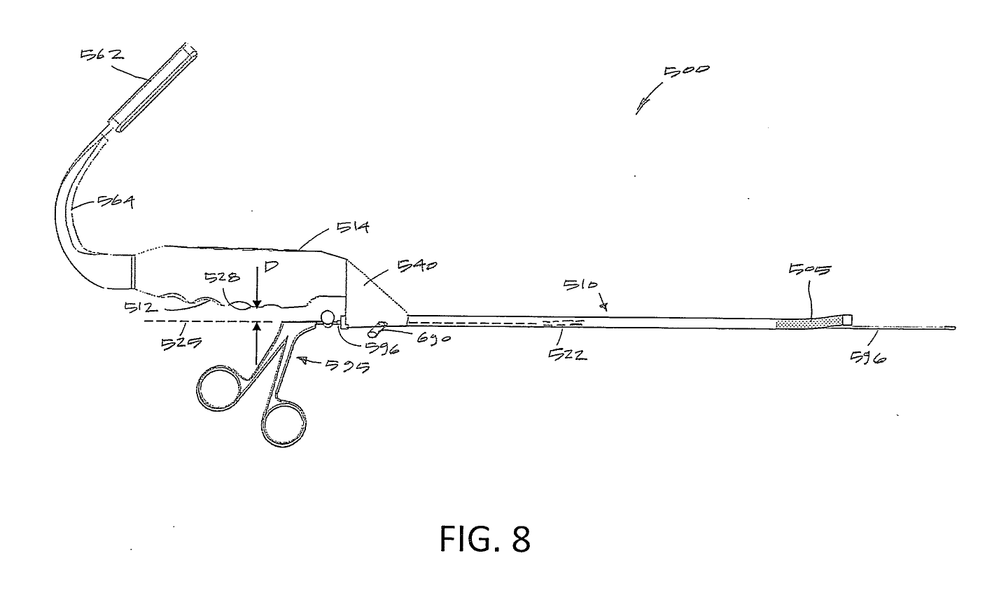

Referring to FIG. 7, it can be seen that the proximal housing or hub 540 of the disposable introducer shaft 510 includes a fitting 542a is adapted for insertion into the receiving recess or fitting 542b of the handle 514. Further, the hub 540 carries at least one electrical connector 545a that is adapted to connect to electrical connector 545b in the handle 514. The electrical connector 545a includes wire leads connectors for the image sensor 550, the LEDs 555, and optionally for pressure sensors, temperature sensors, and flow sensors.

The exploded view of FIG. 7 shows that the display component 560 is detachable from the handle 514. The display component 560 includes the image display or screen 562 and a curved attachment arm 564 that has a male connector portion 565 that is adapted for connection to a recessed connector 566 in the handle 514. In one variation, the male connector portion 565 further carries electrical connector 570 which can be a USB connector for coupling the display 562 to an image processor 575 or components thereof carried within the handle 514. The curved attachment arm 564 can be a rigid molded plastic or a deformable elastomeric material with a deformable core to thus allow the orientation of the display to be adjusted by the user. In one variation, the deformable core 582 is adapted to bend within a predetermined range as indicated in FIG. 7 and also can be twisted within a predetermined range. Further, the display 562 can have a hinge joint or universal joint 584 that couples it to the attachment arm 564. Such a hinge for universal joint 584 can allow for further adjustment of the angle of the display 562 by the user.

FIG. 7 further shows that the handle 514 is adapted for use without attachment of the display component 560. In one variation, the Bluetooth transmitter 588 in the handle 514 can transmit image data to a receiver 590 that in turn is coupled to a remote display 592. In yet another variation, a cable 594 (phantom view) can be plugged into the connector 566 in the proximal end of the handle 514 to send image data to remote display 592.

FIG. 8 is a side view of the endoscope 500 with a biopsy tool 595 having shaft 596 introduced through the working channel 522. It can be seen that the elastomeric tip 505 of the endoscope 500 is deflected as described above. FIG. 8 further illustrates the dimension D by which the central axis 525 of the working channel 522 is offset from the inferior surface 528 of the grip portion 512 of handle 514.

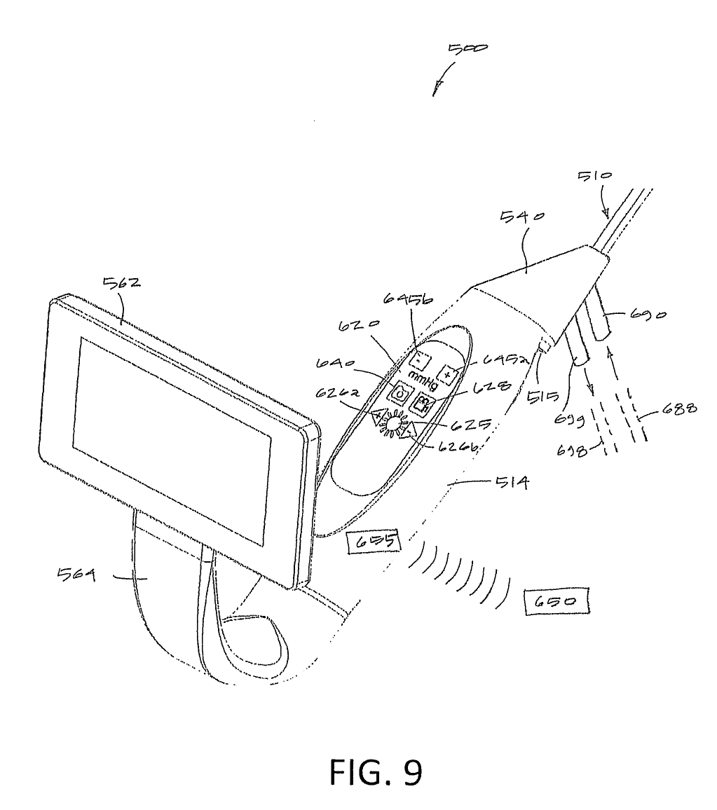

Now turning to FIG. 9, a control pad 620 carried by the handle 514 is shown. The control pad 620 has a first actuator indicated at 625 which can comprise touchpad buttons 626a and 626b (or push buttons that can be depressed) that are adapted to increase or decrease light output from the LEDs 555 in the working end of the endoscope 500. Another actuator 628 is adapted to start and stop video recording with the image sensor 550. Another actuator 640 can be used to store the video recordings or take snapshots for such storage. Finally, actuator buttons 645a and 645b are adapted to increase or decrease fluid pressure in the patient's uterine cavity by communication with a fluid management system 650 that is further described below. In one embodiment, signals from the actuator buttons 645a and 645b are sent by Bluetooth transmitter 655 in the handle 514 to the fluid management system 650 describe further below. Alternatively, an electrical cable may connect the actuator buttons 645a and 645b to the fluid management system.

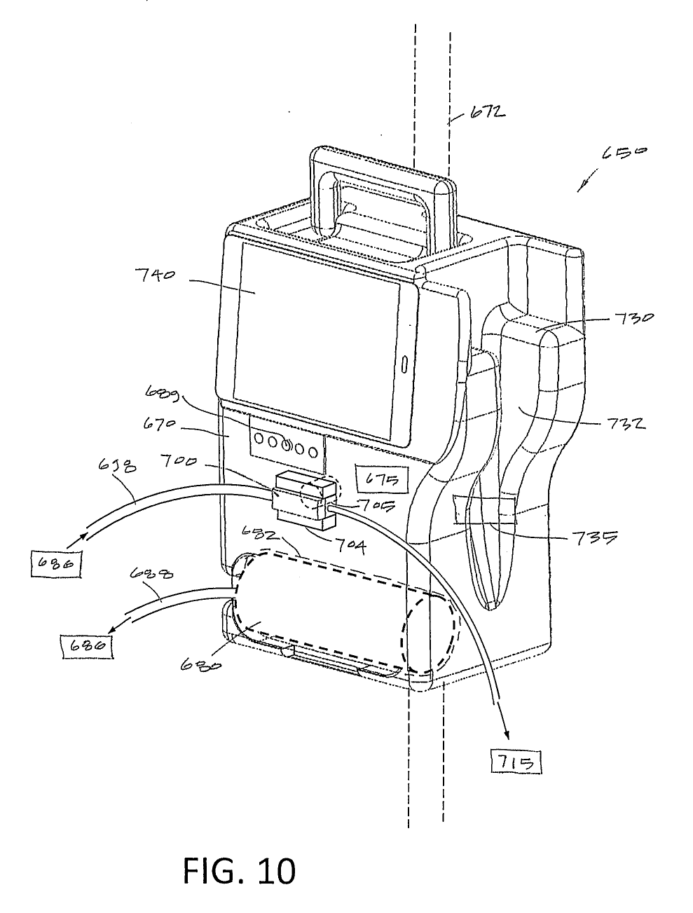

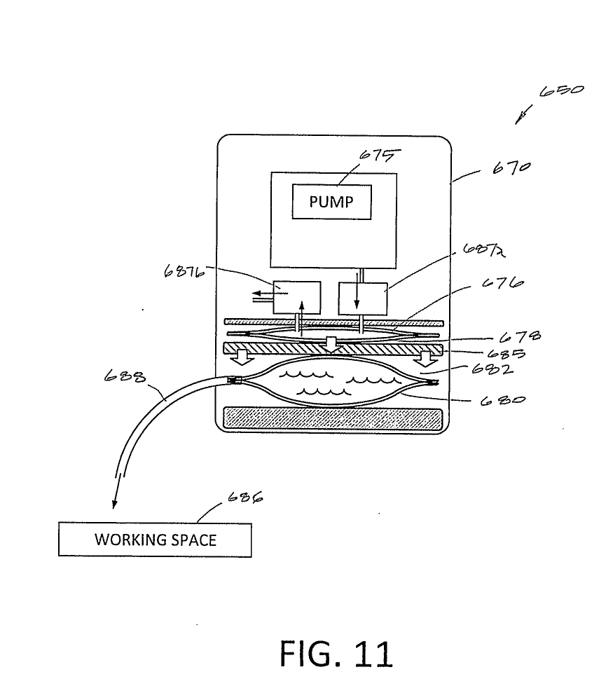

FIGS. 10-11 illustrate a fluid management system 650 of the invention. It can be seen that the fluid management system includes a housing 670 that is carried on a stand with a vertical pole 672 as is known in the art. Within the housing 670 is a pump mechanism 675 that is adapted to expand a bladder 676 with a fluid, such as a gas (see FIG. 11). The walls 678 of the bladder 676 expand to apply pressure on the surface of a fluid-filled sac or container 680 that is carried in a space 682 in the lower portion of housing 670.

Referring to FIG. 11, in one variation, the bladder 676 is adapted to expand against a movable wall 685 which presses against the fluid-filled sac or container 680. Typically, the fluid-filled sac is a bag filled with saline as is known in the art. The saline out outflow provides for irrigation or fluid expansion of a working space 686, such as a patient's uterine cavity. FIG. 11 also shows an inflow control valve 687a intermediate the pump 675 and the bladder 676 for controlling or maintaining fluid pressure in the bladder 676. FIG. 11 further shows an relief valve 687b that is adapted for immediate pressure release from the bladder 676 which can be opened by an software algorithms in response to signals from the pressure sensor 705 or my manual actuation by means of an switch 689 on the housing 670 (see FIG. 10).

In one variation, the saline-filled sac 680 has an outflow line 688 that extends to a port 690 in the hub 540 of the disposable introducer shaft 510 of FIGS. 6 to 9. Thus, the fluid management system 650 can provide fluid inflows into the working space 686 through the introducer shaft 510 of FIGS. 6 and 7. It can be understood that the introducer shaft 510 has an inflow channel therein that carries the fluid inflow to a distal open termination of the inflow channel to reach the targeted working space 686. Further, the introducer shaft 510 as a second outflow channel therein which is adapted to provide for fluid outflows from the working space through an outflow line 698 coupled to a port 699 in the introducer shaft hub 540 (see FIGS. 6 to 9).

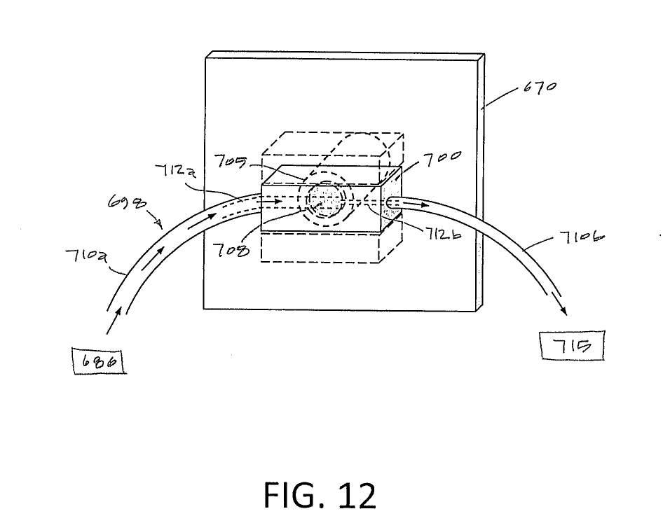

In FIGS. 10-12, it can be seen that the fluid outflow line 698 extends to a outflow fitting 700 that can interlocked with a sensor fitting 704 and pressure sensor 705 carried in the wall of housing 670 of the fluid management system 650. The fitting 700 has thin-wall flexible membrane 708 that interfaces with a non-disposable pressure sensor 705 (see FIG. 12). Thus, the fluid flow in the outflow line 698 can be used to measure fluid pressure in a working space 686, such as a patient's uterine cavity.

FIG. 12 is a schematic view of the outflow fitting 700 which illustrates that the outflow line 698 has a first portion 710a that extends from the working space 686 to the fitting 700 and a second portion 710b that extends from the outflow fitting 700 to a collection reservoir 715. It can be seen that the cross-section of the lumen 712a in the first tubing portion 710a is substantially larger than the cross-section of the lumen 712b in the second tubing portion 710b. This differential cross-section of lumens in the first and second portions of outflow line 698 allows for accurate reading of fluid pressure in the working space. As can be understood, any build up of pressure or drop in pressure in the working space will be sensed immediately by the sensor 705 since the fluid volume in the first portion 710a is substantial and fluid flows rapidly to the outflow fitting 700. This allows for accurate pressure sensing as the pressure in the working space changes, and the lesser cross-section in lumen 712b in second tubing portion 710b is restrictive and thus cannot quickly overcome the pressure changes at the outflow fitting 700 and membrane 708 that interfaces with the pressure sensor 705.

Returning to FIG. 10, it can be seen that the housing 670 further carries a bracket 730 which is adapted to receive the handle 514 of the endoscope device of FIG. 6. The bracket 730 has a receiving portion 732 that can be locked against the device handle 514 and the housing 670 carries a charging station 735 for inductively re-charging a battery 736 carried in handle 614 of the device 500 (see FIGS. 6-7). Thus, the battery 636 can be recharged simply by placing the device handle 514 into the bracket 730 of the housing 670.

Still referring to FIG. 10, it can be seen that the housing 670 further carries an image display 740 for displaying images from the endoscope 500 and/or pressure or other operating parameters of the system. The display 740 further can be detached from the housing and positioned in a different location for the convenience of the physician. In one variation display can communicate with a housing 670 wirelessly or by a cable connection.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. An endoscope system comprising:
(a) an endoscope shaft assembly comprising:
(b) a shaft having a working channel, an inflow channel configured to be coupled to a fluid source, and an outflow channel configured to be coupled to a negative pressure source therethrough,
(c) a hub coupled to a proximal portion of the shaft, and
an image sensor disposed on a distal portion of the shaft;
a control unit configured to adjust a fluid inflow from the fluid source through the inflow channel to the working space and fluid outflow to the negative pressure source through the outflow channel from the working space; and
a handle assembly detachably connectable to the hub of the endoscope shaft assembly, said handle assembly comprising a control pad having at least one actuator linked to the controller for adjusting fluid inflows and outflows through the inflow channel and outflow channel in the shaft.

2. The endoscope system of claim 1 wherein said at least one actuator is wirelessly linked to the controller.

3. The endoscope system of claim 1 wherein said at least one actuator is linked to the controller by a wired connection.

4. The endoscope system of claim 1 further comprising an image display coupled to the handle assembly.

5. The endoscope system of claim 4 wherein said image display is detachably coupled to the handle assembly.

6. The endoscope system of claim 4 further comprising an image processor.

7. The endoscope system of claim 6 wherein the image processor is disposed in the control unit and is electronically coupled to both the image sensor in the shaft assembly and the image display in the handle assembly.

8. The endoscope system of claim 6 wherein the image processor is disposed in the handle assembly and is electronically coupled to both the image sensor in the shaft assembly and the image display in the handle assembly.

9. The endoscope system of claim 4 further comprising a second image display on the control unit.

10. The endoscope system of claim 1 wherein the hub has a first port for detachable connection to the fluid source and a second port for detachable connection to the negative pressure source, whereby no fluids flow through the handle.

11. The endoscope system of claim 10 further comprising one or more tubular connectors for detachably connecting the first and second ports on the hub to the control unit, wherein the control unit comprises a first peristaltic pump operatively connected to the fluid source for delivering fluid inflows to the to the inflow channel and a second peristaltic pump for aspirating fluid outflows from the outflow channel.

12. The endoscope system of claim 1 further comprising a pressure sensor.

13. The endoscope system of claim 12 wherein the pressure sensor is disposed within the endoscope component assembly.

14. The endoscope system of claim 12 wherein the pressure sensor is operatively connected to a flow path between the fluid source and the inflow channel in the shaft.

15. The endoscope system of claim 12 wherein the pressure sensor is operatively connected to a flow path between the outflow channel in the shaft and the negative pressure source.

16. The endoscope system of claim 1 wherein the distal portion of the shaft carries at least one illumination element.

17. The endoscope system of claim 16 wherein the at least one illumination element comprises at least one LED.

18. The endoscope system of claim 16 wherein at least one actuator in the handle assembly is configured to adjust light intensity of the at least one LED.

19. The endoscope system of claim 1 wherein the controller is configured to maintain fluid pressure at a set pressure in the working space.

20. The endoscope system of claim 19 wherein at least one actuator in the handle assembly is configured to adjust the set pressure.

21. The endoscope system of claim 1 wherein at least one actuator in the handle assembly is configured to capture still video images from the image sensor.

22. The endoscope system of claim 1 wherein at least one actuator in the handle assembly is configured to capture video clips from the image sensor.

23. The endoscope system of claim 17 further comprising a video processor in the handle component for processing video signals from the image sensor.

24. The endoscope system of claim 23 further comprising a first electrical connector in the hub that is adapted for detachable coupling to a second connector in the handle component for carrying video and control signals.

25. The endoscope system of claim 24 further comprising a third electrical connector in the hub that is adapted for detachable coupling to a fourth connector in the handle component for connecting an electrical source to the at least one LED.
